(19) United States
(12) Patent Application Publication
(10) Pub. No.: US 2008/0004296 A1
(43) Pub. Date: Jan. 3, 2008

(54) Organic Compounds

(76) Inventors: Christine Charman, West Sussex (GB); Alyson Fox, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/570,393

(22) PCT Filed: Jun. 16, 2005
(86) PCT No.: PCT/EP05/06494

§ 371 Date: Mar. 19, 2007

(30) Foreign Application Priority Data
Jun. 17, 2004 (GB) 0413618.0

(51) Int. Cl. A61K 31/517; A61P 11/14

(52) U.S. Cl. 514/266.3

(57) ABSTRACT

The use of a compound of formula I 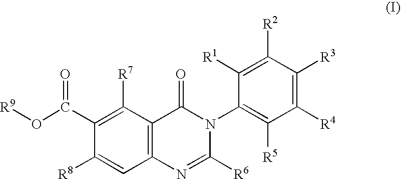
in free base or acid addition salt form for the preparation of a medicament for the treatment of cough, wherein R¹, R², R³, R⁴, R⁵, R⁶, R⁷, R⁸ and R⁹ have the meanings as indicated in the specification.

This invention relates to organic compounds and, in particular to their use as pharmaceuticals.

The invention provides, in one aspect, use of a compound of formula I 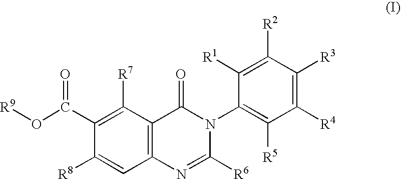
in free base or acid addition salt form for the preparation of a medicament for the treatment of cough, wherein

R¹ R², R³, R⁴ and R⁵ independently are hydrogen; halogen; C₁-C₄alkyl; C₂-C₄alkenyl; C₃-C₇cycloalkyl; C₃-C₇cycloalkylC₁-C₄alkyl; C₁-C₄alkoxyC₁-C₄alkyl; C₁-C₄alkylcarboxy;

hydroxyC₁-C₄alkoxyC₁-C₄alkyl; hydroxy; hydroxyC₁-C₄alkyl; phenylC₁-C₄alkyl which is optionally substituted by hydroxy, C₁-C₄alkoxy, carboxy, C₁-C₄alkoxycarbonylC₁-C₄alkyl, C₁-C₄alkoxycarbonyl, cyano; —SO₂R¹⁰; cyano; —SO₂N(R¹⁰)R¹¹;—S—R¹⁰ or —SOR¹⁰; or R¹ and R² or R² and R³ denote, together with the carbon atoms to which they are attached, an aromatic or aliphatic carbocyclic group having 5 to 10 ring atoms or a heterocyclic group having 5 to 10 ring atoms of which one, two or three are hetero atoms selected from nitrogen, oxygen and sulfur;

R⁶ is —CH₂—O—C(O)—N(R¹²)R¹³, —CH₂—X—C(O)—R¹⁴, C₁-C₄alkyl or hydroxyC₁-C₄alkyl;

R⁷, R⁸ and R⁹ independently are C₁-C₄alkyl;

R¹⁰ and R¹¹ independently are hydrogen, C₁-C₄alkyl; C₂-C₄alkenyl; C₃-C₇cycloalkyl; C₃-C₇cycloalkylC₁-C₄alkyl; C₁-C₄alkoxyC₁-C₄alkyl; C₁-C₄alkylcarboxy; hydroxyC₁-C₄alkoxyC₁l-C₄alkyl; hydroxy; hydroxyC₁-C₄alkyl; phenylC₁-C₄alkyl which is optionally substituted by hydroxy, C₁-C₄alkoxy, carboxy, C₁-C₄alkoxycarbonylC₁-C₄alkyl, C₁-C₄alkoxycarbonyl, cyano; or R¹⁰ and R¹¹ together form a heterocyclic group having 5 to 10 ring atoms of which one, two or three are hetero atoms selected from nitrogen, oxygen and sulfur; R¹² and R¹³ independently are hydrogen, C₁-C₄alkyl, C₂-C₄alkenyl, C₃-C₇cycloalkyl, C₃-C₇cycloalkylC₁-C₄alkyl, C₁-C₄alkoxyC₁-C₄alkyl, hydroxyC₁-C₄alkoxyC₁-C₄alkyl, hydroxyC₁-C₄alkyl, dihydroxyC₁-C₄alkyl, C₁-C₄alkoxycarbonylC₁-C₄alkyl, C₁-C₄alkoxycarbonyl, cyano, —SO₂R¹⁰, —SO₂N(R¹⁰)R¹¹, —S—R¹⁰, —SOR¹⁰, —C₁-C₄-alkylene-SO₂R¹⁰, —C₁-C₄-alkylene-SOR¹⁰, —C₁-C₄-alkylene-NH—SO₂R¹⁰, —Cₗ-C₄-alkylene-CON(R¹⁰)R¹¹, —CON(R¹⁰)R¹¹, —C₁-C₄-alkylene-C(O)OR¹⁰,fluoroalkyl, or R¹² and R¹³ form a substituted or unsubstituted heterocyclic group having 5 to 10 ring atoms;

R¹⁴ is NH, C₁-C₄alkyl-NH—, C₂-C₄alkenyl-NH—, C₃-C₇cycloalkyl-NH—, C₃-C₇cycloalkylC₁-C₄alkyl-NH—, C₁-C₄alkoxyC₁-C₄alkyl-NH—, hydroxyC₁-C₄alkoxyC₁-C₄alkyl-NH—, hydroxyC₁-C₄alkyl-NH—, dihydroxyC₁-C₄alkyl-NH—, C₁-C₄alkoxycarbonylC₁-C₄alkyl-NH—, C₁-C₄alkoxycarbonyl-NH—, —NH—C₁-C₄-alkylene-CN, —NH—SO₂R¹⁰, —NH—SO₂N(R¹⁰)R¹¹, —NH—C₁-C₄-alkylene-S—R¹⁰, —NH—SOR¹⁰, —NH—C₁-C₄-alkylene-SO₂R¹⁰, —NH—C₁-C₄-alkylene-SOR¹⁰, —NH—C₁-C₄-alkylene-NH—SO₂R¹⁰, —NH—C₁-C₄-alkylene-CON(R¹⁰)R¹¹, —NH—CON(R¹⁰)R¹¹, —NH—C₁-C₄-alkylene-C(O)OR¹⁰, —NH-fluoroalkyl, or a substituted or unsubstituted heterocyclic group having 5 to 10 ring atoms; and

X is O or CH₂.

The invention provides, in another aspect, a method of treating cough, in a subject, particularly a human subject, in need of such treatment, which comprises administering to said subject an effective amount of a compound of formula I as hereinbefore defined.

Treatment of cough in accordance with the invention may be symptomatic.

Cough to be treated in accordance with the invention includes conditions associated with an action that occurs as the result of irritation of the throat, larynx and airways. It takes the form of a noisy expulsion of air, and its function is to clear the airways of the substances causing the irritation anything from particles of dust to smoke, chemicals, and virus-laden mucus. Cough may be due to one or more underlying diseases including chronic obstructive pulmonary disease (COPD), upper respiratory tract infections (URTI), postnasal drip, acute viral infection, asthma, lung cancer, interstitial lung disease, gastroesophageal reflux disease (GERD), angiotensin converting enzyme-induced cough and heart failure. The compounds of the formula I are effective in treating cough including non-productive cough, also known as dry cough, particularly non-productive cough associated with COPD or acute viral infection.

The terms used in the present specification have the following meanings:

“Optionally substituted” as used herein means the group referred to can be substituted at one or more positions by any one or any combination of the radicals listed thereafter.

“Halo” or “halogen” as used herein denotes a element belonging to group 17 (formerly group VII) of the Periodic Table of Elements, which may be, for example, fluorine, chlorine, bromine or iodine. Preferably halo or halogen is fluorine or chlorine.

“C₁-C₄-alkyl” as used herein denotes straight chain or branched alkyl that contains one to four carbon atoms.

“C₁-C₄-alkylene” as used herein denotes a straight chain or branched alkylene that contains one to four carbon atoms.

“C₂-C₄-alkenyl” as used herein denotes straight chain or branched hydrocarbon chains that contain two to four carbon atoms and one or more carbon-carbon double bonds.

“C₁-C₄-alkoxy” as used herein denotes straight chain or branched alkoxy that contains 1 to 4 carbon atoms.

“Fluoro-C₁-C₄-alkyl”, “hydroxy-C₁-C₄-alkyl” and “phenyl-C_{1-C}₄-alkyl” as used herein denote C₁-C₄-alkyl as hereinbefore defined substituted at one or more positions by fluoro, hydroxy or phenyl respectively.

“C₁-C₄-alkyl-NH—” and “C₂-C₄-alkenyl-NH” as used herein denotes amino substituted by C₁-C₄-alkyl as hereinbefore defined or C₂-C₄-alkenyl as hereinbefore defined respectively.

“C₁-C₄-alkoxy-C₁-C₄-alkyl” as used herein denotes C₁-C₄-alkyl as hereinbefore defined substituted by C₁-C₄-alkoxy.

“C₁-C₄-alkoxycarboxy” and “C₁-C₄-alkoxycarbonyl” as used herein denote C₁-C₄-alkoxy as hereinbefore defined linked through an oxygen atom thereof to a carboxy group or a carbonyl group respectively.

“Hydroxy-C₁-C₄-alkoxy” as used herein denotes C₁-C₄-alkoxy as hereinbefore substituted by hydroxy.

“Hydroxy-C₁-C₄-alkoxy-C₁-C₄-alkyl” as used herein denotes C₁-C₄-alkyl as hereinbefore defined substituted by hydroxy-C₁-C₄-alkoxy as hereinbefore defined through the oxygen atom of the C₁-C₄-alkoxy group thereof.

“Carbocyclic group having 5 to 10 ring atoms” as used herein denotes a carbocyclic group having 5 to 10 ring carbon atoms, which can be aliphatic such as a C₅-C₇-cycloalkyl, or aromatic, such as phenyl, and in either case is optionally substituted by one or more, usually one or two, C₁-C₄-alkyl groups.

“C₃-C₇-cycloalkyl” as used herein denotes cycloalkyl having 3 to 7 ring carbon atoms, for example a monocyclic group such as a cyclopropyl, cyclobutyl, cyclopentyl, cyclohexyl or cycloheptyl, any of which can be substituted by one or more, usually one or two, C₁-C₄-alkyl groups, or a bicyclic group such as bicycloheptyl. Preferably C₃-C₇-cycloalkyl is C₄-C₆-cycloalkyl, for example cyclobutyl, cyclopentyl or cyclohexyl.

“C₃-C₇-cycloalkyl-C₁-C₄-alkyl” as used herein denotes C₁-C₄-alkyl as hereinbefore defined substituted by C₃-C₇-cycloalkyl as hereinbefore defined.

“Heterocyclic group having 5 to 10 ring atoms of which one, two or three are hetero atoms selected from nitrogen, oxygen and sulfur” as used herein may be for example, pyrrole, pyrrolidine, pyrazole, imidazole, triazole, tetrazole, thiadiazole, oxazole, isoxazole, thiophene, thiazole, isothiazole, oxadiazole, pyridine, pyrazine, pyridazine, pyrimidine, piperidine, piperazine, triazine, oxazine, morpholino, quinoline, isoquinoline, naphthyridine, indane or indene. Preferred heterocyclic rings include pyrrolidine and morpholino.

The invention relates to the use of compounds of formula I that preferably include those wherein

R¹ is hydrogen, chloro, methyl, methoxy, —CH₂C(O)OCH₃, —CH₂CH₂C(O)OCH₃, —C(O)N(CH₃)₂, —C(O)OCH₃, cyano, —SO₂-1-pyrrolidinyl, —SO₂CH₃, —SO₂NHCH₃, —SO₂N(CH₃)₂, —SO₂N(CH₃)CH₂COOH, —S—CH₃ or —SOCH₃,

R² is hydrogen, chloro, methyl or tri-fluoromethyl, and

R³ is hydrogen, fluoro, methyl,

or R¹ and R² form a —NH—CH₂—CH₂—CH₂— ring or a —CH═CH—CH═CH— ring,

or R² and R³ form a —CH═CH—CH═CH— ring;

R⁴ is hydrogen or chloro;

R⁵ is hydrogen or chloro;

R⁶ is methyl, hydroxymethyl, —CH₂—O—C(O)—N(R¹²)R¹³ or —CH₂—X—C(O)—R¹⁴;

R⁷ and R³ are methyl;

R⁹ is ethyl or propyl;

R¹⁰ is 1-pyrrolidinyl, —CH₂COOH, methyl or hydrogen;

R¹¹ is methyl or hydrogen;

R¹² is methyl, ethyl, propyl, 2-hydroxyethyl, 3-hydroxypropyl, 2,3 dihydroxypropyl,

1-(hydroxymethyl)-2-hydroxyethyl, cyano, —CH₂CH₂—SO₂CH₃, —CH₂CH₂—S—CH₃, —CH₂CH₂—NH—SO₂CH₃, —CH₂C(O)OCH₃, —CH₂CONH₂, 2,2,2-trifluoro-ethyl, and R¹³ is hydrogen,

or R¹² and R¹³ form a —CH₂—CH₂—CHOH—CH₂—CH₂— ring;

R¹⁴ is —NH—CH₃, —NH—CH₂CH₃, —NH—CH₂CH₂CH₃, 2-hydroxyethyl-NH—,

3-hydroxypropyl-NH—, 2,3 dihydroxypropyl-NH—, 1-(hydroxymethyl)-2-hydroxyethyl-NH—, —NH—CH₂CN, —NH—CH₂CH₂—SO₂CH₃, —NH—CH₂CH₂—S—CH₃, —NH—CH₂CH₂—NH—SO₂CH₃, —NH—CH₂C(O)OCH₃, —NH—CH₂CONH₂, 2,2,2-NH-trifluoro-ethyl, 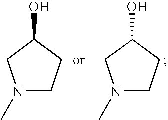
and

X is O.

The invention relates to the use of compounds of formula I that especially include those wherein

R¹ is —SO₂NHCH₃;

R² is hydrogen;

R³ is hydrogen and chloro;

R⁴ is hydrogen;

R⁵ is hydrogen;

R⁶ is methyl, hydroxymethyl, —CH₂—O—C(O)—N(R¹²)R¹³ or —CH₂—X—C(O)—R¹⁴;

R⁷ and R⁸ are both methyl;

R⁹ is ethyl;

R¹⁰ is methyl;

R¹¹ is hydrogen;

R¹² is ethyl or 2-hydroxyethyl and R¹³ is hydrogen,

or R¹² and R¹³ form a —CH₂—CH₂—CHOH—CH₂—CH₂— ring;

R¹⁴ is 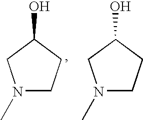
or 2-hydroxyethyl-NH—; and

X is O.

Compounds of the invention exist in free base or acid addition salt form. The invention is to be understood as including the compounds of formula I in free as well as in acid addition salt form, e.g. as trifluoroacetate or hydrochloride salt. Suitable pharmaceutically acceptable acid addition salts for pharmaceutical use in accordance with the invention include in particular the hydrochloride salt.

Especially preferred examples of compounds of formula I include:

2-Ethylcarbamoyloxymethyl-5,7-dimethyl-3-(2-methylsulfamoyl-phenyl)-4-oxo-3,4-dihydro-quinazoline-6-carboxylic acid ethyl ester;

2-(2-Hydroxy-ethylcarbamoyloxymethyl)-5,7-dimethyl-3-(2-methylsulfamoyl-phenyl)-4-oxo-3,4-dihydro-quinazoline-6-carboxylic acid ethyl ester;

3-(2-Chlorophenyl)-2-(2-ethylcarbamoylethyl)-5,7-dimethyl-4-oxo-3,4-dihydroquinazoline-6-carboxylic acid ethyl ester;

2-(2-Hydroxy-ethylcarbamoyloxymethyl)-5,7-dimethyl-3-(2-methylsulfamoylphenyl)-4-oxo-3,4-dihydro-quinazoline-6-carboxylic acid propyl ester;

2-Hydroxymethyl-5,7-dimethyl-3-(2-methylsulfamoyl-phenyl)-4-oxo-3,4-dihydroquinazoline-6-carboxylic acid butyl ester; and

2,5,7-Trimethyl-3-(2-methylsulfamoylphenyl)-4-oxo-3,4-dihydro-quinazoline-6-carboxylic acid cyclobutylmethyl ester.

The invention relates most importantly to the use of 2-(2-Hydroxy-ethylcarbamoyloxy-methyl)-5,7-dimethyl-3-(2-methylsulfamoyl-phenyl)-4-oxo-3,4-dihydro-quinazoline-6-carboxylic acid ethyl ester, i.e. a compound of formula II

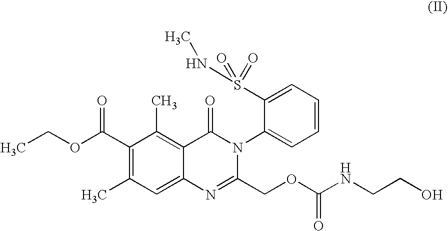

The compounds of formula I, in free base or acid addition salt form, may be prepared as described in WO 031066603, the contents of which is incorporated herein by reference.

Depending upon the nature of the variables and the corresponding number of centres of asymmetry and also upon the starting materials and procedures chosen, the compounds of formula I may be obtained in the form of mixtures of stereoisomers, for example mixtures of diastereoisomers or mixtures of enantiomers, such as racemates, or possibly also in the form of pure stereoisomers. Mixtures of diastereoisomers obtainable in accordance with the process or by some other method can be separated in customary manner into mixtures of enantiomers, for example racemates, or into individual diastereoisomers, for example on the basis of the physico-chemical differences between the constituents in known manner by fractional crystallisation, distillation and/or chromatography. Advantageously the more active isomer is isolated.

Mixtures of enantiomers, especially racemates, obtainable in accordance with the process or by some other method can be separated into the individual enantiomers by known methods, for example by recrystallisation from an optically active solvent, with the aid of microorganisms, by chromatography and/or by reaction with an optically active auxiliary compound, for example a base, acid or alcohol, to form mixtures of diastereoisomeric salts or functional derivatives, such as esters, separation thereof and freeing of the desired enantiomer. Advantageously the more active enantiomer is isolated.

In the treatment of disorders in accordance with the invention, compounds of formula I, in free form or in pharmaceutically acceptable salt form, may be administered by any appropriate route, for example orally, e.g. in tablet, capsule or liquid form, parenterally, for example in the form of an injectable solution or suspension, or intranasally, for example in the form of an aerosol or other atomisable formulation using an appropriate intranasal delivery device, e.g. a nasal spray such as those known in the art, or by inhalation. Oral administration is however preferred.

A compound of formula I or II in free base or acid addition salt form may be administered in a pharmaceutical composition together with a pharmaceutically acceptable diluent or carrier. Such compositions may be as described in WO 031066603, for example dry powders, tablets, capsules and liquids, but also injection solutions, infusion solutions or inhalation suspensions, which may be prepared using other formulating ingredients and techniques known in the art.

The dosage of the compound of formula I or II in free base or acid addition salt form can depend on various factors, such as the activity and duration of action of the active ingredient, the severity of the condition to be treated, the mode of administration, the species, sex, ethnic origin, age and weight of the subject and/or its individual condition. In a normal case the daily dose for administration, for example oral administration, to a warm-blooded animal, particularly a human being, weighing about 75 kg is estimated to be from approximately 0.7 mg to approximately 1400 mg, especially from approximately 5 mg to approximately 200 mg. That dose may be administered, for example, in a single dose or in several part doses of, for example, from 5 to 200 mg.

The suitability of a compound of formula I for the treatment of cough, for example non-productive cough, may be tested by determining the inhibitory effect of a compound of formula I on cough induced by release of endogenous neuropeptides in guinea pigs using the method described in El-Hashim A. et al. Pulmonary Pharmacology & Therapeutics 17(1) (2004) pages 11-18.

The present invention is illustrated by the following non-limiting Example.

### EXAMPLE

Male Dunkin-Hartley guinea-pigs (350-550 g) are exposed to either air or tobacco smoke generated by four research cigarettes, (2R4F; Kentucky, USA) for a 3 second puff every 30 sec, for 2 days. At 24 hours after the last exposure conscious, unrestrained animals are placed in a plethysmograph chamber (Buxco, USA) and challenged with an aerosol of 0.3 M citric acid in 0.9% saline for 10 minutes. Cough and Penh AUC (measure of bronchoconstriction) are recorded during the 10 minute exposure and for a further 10 minutes after exposure using the method described in El-Hashim A. et al. Pulmonary Pharmacology & Therapeutics 17(1) (2004) pages 11-18.

To evaluate the effects of 2-(2-Hydroxy-ethylcarbamoyloxymethyl)-5,7-dimethyl-3-(2-methylsulfamoyl-phenyl)-4-oxo-3,4-dihydro-quinazoline-6-carboxylic acid ethyl ester on the enhanced cough response, animals are dosed subcutaneously with the compound (0.3-10 mg/kg, n=3-10) or vehicle (3:1 PEG200:saline, n=10) at 1 hour prior to citric acid challenge. The percentage inhibition of the cough response following compound administration compared with vehicle administration in tobacco smoke-exposed animals is determined. Data are expressed as mean±s.e. mean and significance is defined as p<0.05 (ANOVA & Dunnett's multiple comparison test).

Following tobacco smoke exposure a significant increase in the cough response to 0.3 M citric acid is observed at 24 hours after the last exposure, with no effect on Penh. 2-(2-Hydroxy-ethylcarbamoyloxymethyl)-5,7-dimethyl-3-(2-methylsulfamoyl-phenyl)-4-oxo-3,4-dihydro-quinazoline-6-carboxylic acid ethyl ester (0.3, 1, 3 or 10 mg/kg) inhibits tobacco-smoke-enhanced cough by 18±14, 49±17, 80±13 and 100±0%, respectively, reaching significance at 1 to 10 mg/kg. The baseline Penh value is inhibited by 48±17, 55±19, 77±10 and 86±2%, for 0.3, 1, 3, and 10 mg/kg 2-(2-hydroxy-ethylcarbamoyloxymethyl)-5,7-dimethyl-3-(2-methylsulfamoyl-phenyl)-4-oxo-3,4-dihydro-quinazoline-6-carboxylic acid ethyl ester, respectively, demonstrating significant inhibition at 3 mg/kg only.

1-10. (canceled)

11. A method of treating a subject suffering from cough which comprises administering to that subject an effective amount of a compound of formula I 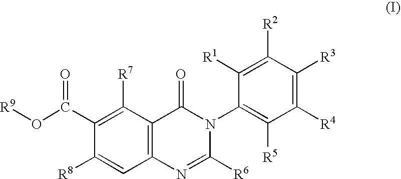
in free base or acid addition salt form, wherein
R¹ R², R³, R⁴ and R⁵ independently are hydrogen; halogen; C₁-C₄alkyl; C₂-C₄alkenyl; C₃-C₇cycloalkyl; C₃-C₇cycloalkylC₁-C₄alkyl; C₁-C₄alkoxyC₁-C₄alkyl; C₁-C₄alkylcarboxy; hydroxyC₁-C₄alkoxyC₁-C₄alkyl; hydroxy; hydroxyC₁-C₄alkyl; phenylC₁-C₄alkyl which is optionally substituted by hydroxy, C₁-C₄alkoxy, carboxy, C₁-C₄alkoxycarbonylC₁-C₄alkyl, C₁-C₄alkoxycarbonyl, cyano; —SO₂R¹⁰; cyano; —SO₂N(R¹⁰)R¹¹; —S—R¹⁰ or —SOR¹⁰; or R¹ and R² or R² and R³ denote, together with the carbon atoms to which they are attached, an aromatic or aliphatic carbocyclic group having 5 to 10 ring atoms or a heterocyclic group having 5 to 10 ring atoms of which one, two or three are hetero atoms selected from nitrogen, oxygen and sulfur;
R⁶ is —CH₂—O—C(O)—N(R¹²)R¹³, —CH₂—X—C(O)—R¹⁴, C₁-C₄alkyl or hydroxyC₁-C₄alkyl;
R⁷, R⁸ and R⁹ independently are C₁-C₄alkyl;
R¹⁰ and R¹¹ independently are hydrogen, C₁-C₄alkyl; C₂-C₄alkenyl; C₃-C₇cycloalkyl; C₃-C₇cycloalkylC₁-C₄alkyl; C₁-C₄alkoxyC₁-C₄alkyl; C₁-C₄alkylcarboxy; hydroxyC₁-C₄alkoxyC₁-C₄alkyl; hydroxy; hydroxyC₁-C₄alkyl; phenylC₁-C₄alkyl which is optionally substituted by hydroxy, C₁-C₄alkoxy, carboxy, C₁-C₄alkoxycarbonylC₁-C₄alkyl, C₁-C₄alkoxycarbonyl, cyano; or R¹⁰ and R¹¹ together form a heterocyclic group having 5 to 10 ring atoms of which one, two or three are hetero atoms selected from nitrogen, oxygen and sulfur;
R¹² and R¹³ independently are hydrogen, C₁-C₄alkyl, C₂-C₄alkenyl, C₃-C₇cycloalkyl, C₃-C₇cycloalkylC₁-C₄alkyl, C₁-C₄alkoxyC₁-C₄alkyl, hydroxyC₁-C₄alkoxyC₁-C₄alkyl, hydroxyC₁-C₄alkyl, dihydroxyC₁-C₄alkyl, C₁-C₄alkoxycarbonylC₁-C₄alkyl, C₁-C₄alkoxycarbonyl, cyano, —SO₂R¹⁰, —SO₂N(R¹⁰)R¹¹, —S—R¹⁰, —SOR¹⁰, —C₁-C₄-alkylene-SO₂R¹⁰, —C₁-C₄-alkylene-SOR¹⁰, —C₁-C₄-alkylene-NH—SO₂R¹⁰, —C₁-C₄-alkylene-CON(R¹⁰)R¹¹, —CON(R¹⁰)R¹¹, —C₁-C₄-alkylene-C(O)OR¹⁰,fluoroalkyl, or R¹² and R¹³ form a substituted or unsubstituted heterocyclic group having 5 to 10 ring atoms;
R¹⁴ is NH, C₁-C₄alkyl-NH—, C₂-C₄alkenyl-NH—, C₃-C₇cycloalkyl-NH—, C₃-C₇cycloalkylC₁-C₄alkyl-NH—, C₁-C₄alkoxyC₁-C₄alkyl-NH—, hydroxyC₁-C₄alkoxyC₁-C₄alkyl-NH—, hydroxyC₁-C₄alkyl-NH—, dihydroxyC₁-C₄alkyl-NH—, C₁-C₄alkoxycarbonylC₁-C₄alkyl-NH—, C₁-C₄alkoxycarbonyl-NH—, —NH—C₁-C₄-alkylene-CN, —NH—SO₂R¹⁰, —NH—SO₂N(R¹⁰)R¹¹, —NH—C₁-C₄-alkylene-S—R¹⁰, —NH—SOR¹⁰, —NH—C₁-C₄-alkylene-SO₂R¹⁰, —NH—C₁-C₄-alkylene-SOR¹⁰, —NH—C₁-C₄-alkylene-NH—SO₂R¹⁰, —NH—C₁-C₄-alkylene-CON(R¹⁰)R¹¹, —NH—CON(R¹⁰)R¹¹, —NH—C₁-C₄-alkylene-C(O)OR¹⁰, —NH-fluoroalkyl, or a substituted or unsubstituted heterocyclic group having 5 to 10 ring atoms; and
X is O or CH₂;
with the proviso that when R¹ is either halogen, methyl, ethyl, methoxy, trifluoromethyl or hydrogen and R², R³, R⁴ are either hydrogen, methyl or methoxy and R⁵ is hydrogen or methyl, R¹² is neither hydrogen, C₂-C₄alkyl, C₂-C₄alkenyl, hydroxyC₁-C₄alkyl, —C₁-C₄-alkylene-SO₂R¹⁰, nor —C₁-C₄-alkylene-SOR¹⁰.

12. A method according to claim 11, wherein
R¹ is hydrogen, chloro, methyl, methoxy, —CH₂C(O)OCH₃, —CH₂CH₂C(O)OCH₃, —C(O)N(CH₃)₂, —C(O)OCH₃, cyano, —SO₂-1-pyrrolidinyl, —SO₂CH₃, —SO₂NHCH₃, —SO₂N(CH₃)₂, —SO₂N(CH₃)CH₂COOH, —S—CH₃ or —SOCH₃,
R² is hydrogen, chloro, methyl or tri-fluoromethyl, and
R³ is hydrogen, fluoro, methyl,
or R¹ and R² form a —NH—CH₂—CH₂—CH₂— ring or a —CH═CH—CH═CH— ring,
or R² and R³ form a —CH═CH—CH═CH— ring;
R⁴ is hydrogen or chloro;
R⁵ is hydrogen or chloro;
R⁶ is methyl, hydroxymethyl, —CH₂—O—C(O)—N(R¹²)R¹³ or —CH₂—X—C(O)—R¹⁴;
R⁷ and R⁸ are methyl;
R⁹ is ethyl or propyl;
R¹⁰ is 1-pyrrolidinyl, —CH₂COOH, methyl or hydrogen;
R¹¹ is methyl or hydrogen;
R¹² is methyl, ethyl, propyl, 2-hydroxyethyl, 3-hydroxypropyl, 2,3 dihydroxypropyl,
1-(hydroxymethyl )-2-hydroxyethyl, cyano, —CH₂CH₂—SO₂CH₃, —CH₂CH₂—S—CH₃, —CH₂CH₂—NH—SO₂CH₃, —CH₂C(O) OCH₃,—CH₂CONH₂, 2,2,2-trifluoro-ethyl, and R¹³ is hydrogen,
or R¹² and R¹³ form a —CH₂—CH₂—CHOH—CH₂—CH₂— ring;
R¹⁴ is —NH—CH₃, —NH—CH₂CH₃, —NH—CH₂CH₂CH₃, 2-hydroxyethyl-NH—,
3-hydroxypropyl-NH—, 2,3dihydroxypropyl-NH—, 1-(hydroxymethyl )-2-hydroxyethyl-NH—, —NH—CH₂CN, —NH—CH₂CH₂—SO₂CH₃, —NH—CH₂CH₂—S—CH₃, —NH—CH₂CH₂—NH—SO₂CH₃, —NH—CH₂C(O)OCH₃, —NH—CH₂CONH₂, 2,2,2-NH-trifluoro-ethyl, 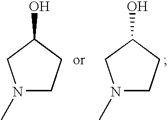
and
X is O.

13. A method according to claim 12, wherein
R¹ is —SO₂NHCH₃;
R² is hydrogen;
R³ is hydrogen and chloro;
R⁴ is hydrogen;
R⁵ is hydrogen;
R⁶ is methyl, hydroxymethyl, —CH₂—O—C(O)—N(R¹²)R¹³ or —CH₂—X—C(O)—R¹⁴;
R⁷ and R⁸ are both methyl;
R⁹ is ethyl;
R¹⁰ is methyl;
R¹¹ is hydrogen;
R¹² is ethyl or 2-hydroxyethyl and R¹³ is hydrogen,
or R¹² and R¹³ form a —CH₂—CH₂—CHOH—CH₂—CH₂— ring;
R¹⁴ is 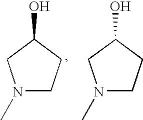
or 2-hydroxyethyl-NH—; and
X is O.

14. A method according to claim 13, wherein the compound of formula I is selected from the group consisting of:
2-ethylcarbamoyloxymethyl-5,7-dimethyl-3-(2-methylsulfamoyl-phenyl) -4-oxo-3,4-dihydro-quinazoline-6-carboxylic acid ethyl ester;
2-(2-Hydroxy-ethylcarbamoyloxymethyl)-5,7-dimethyl-3-(2-methylsulfamoyl-phenyl) -4-oxo-3,4-dihydro-quinazoline-6-carboxylic acid ethyl ester;
3-(2-Chlorophenyl )-2-(2-ethylcarbamoylethyl )-5,7-dimethyl-4-oxo-3,4-dihydroquinazoline-6-carboxylic acid ethyl ester;
2-(2-Hydroxy-ethylcarbamoyloxymethyl)-5,7-dimethyl-3-(2-methylsulfamoylphenyl)-4-oxo-3,4-dihydro-quinazoline-6-carboxylic acid propyl ester;
2-Hydroxymethyl-5,7-dimethyl-3-(2-methylsulfamoyl-phenyl )-4-oxo-3,4-dihydroquin-azoline-6-carboxylic acid butyl ester; and
2,5,7-Trimethyl-3-(2-methylsulfamoylphenyl)-4-oxo-3,4-dihydro-quinazoline-6-carboxylic acid cyclobutylmethyl ester.

15. A method according to claim 14, wherein the compound of formula I is a compound of formula II

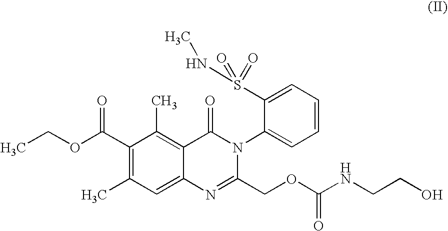

16. A method according to claim 11, in which the cough is a productive cough.

17. A method according to claim 11, in which the cough is a non-productive cough.

18. A method according to claim 17, in which the cough is associated with chronic obstructive pulmonary disease or acute viral infection.

19. A method according to claim 17, in which the cough is associated with asthma.
